(19) United States
(12) Patent Application Publication
(10) Pub. No.: US 2018/0327345 A1
(43) Pub. Date: Nov. 15, 2018

(54) PRODUCTION OF ESTERS OF 3-HYDROXYPROPIONIC ACID AND ACRYLIC ACID

(71) Applicant: BASF SE, Ludwigshafen Am Rhein (DE)

(72) Inventors: Marek PAZICKY, Ludwigshafen (DE); Rocco PACIELLO, Ludwigshafen (DE); David LINDER, Freiburg (DE); Mathieu BLANCHOT, Ludwigshafen (DE); Wolfgang FISCHER, Ludwigshafen (DE)

(21) Appl. No.: 15/775,938

(22) PCT Filed: Nov. 16, 2016
(86) PCT No.: PCT/EP2016/077851

§ 371 Date: May 14, 2018

Related U.S. Application Data
(60) Provisional application No. 62/256,161, filed on Nov. 17, 2015.

(30) Foreign Application Priority Data
Nov. 17, 2015 (DE) 10 2015 222 675.1

(51) Int. Cl. C07C 67/327; C07C 67/03

(52) U.S. Cl. CPC C07C 67/327; C07C 67/03

(57) ABSTRACT

3-Hydroxypropionic acid esters are obtained by reacting ethylene oxide with carbon monoxide in the presence of a cobalt catalyst, wherein poly-3-hydroxypropionate is obtained; and transesterification of the poly-3-hydroxypropionate with an alcohol in the presence of a transesterification catalyst, wherein the 3-hydroxypropionic acid ester is obtained. The transesterification catalyst is a compound of the formula MLₓ, where M is a metal of main groups 2, 3 or 4 or transition groups 3 to 8 of the Periodic Table of the Elements, L is a ligand which is bonded directly to M via a C, an O, a P, an S and/or an N atom, and x is an integer from 2 to 6.

Acrylic esters are important intermediates, the main application of which is in the production of homo- and copolymers with e.g. acrylic acid, acrylamides, methacrylates, acrylonitrile, maleates, vinyl acetate, vinyl chloride, styrene, butadiene and unsaturated polyesters. These polymers can be used, for example, as polymer dispersions for adhesives and sealants, lacquers and paints, coatings for textiles, leather, paper and also for numerous plastics. Produced in large quantities are, inter alia, methyl acrylate, ethyl acrylate, butyl acrylate, isobutyl acrylate and 2-ethylhexyl acrylate.

Acrylic esters are currently produced industrially by the oxidation of propene to acrylic acid and subsequent esterification. Propene is principally obtained by thermal cleavage (steam cracking) of longer-chain alkanes from fossil raw materials such as petroleum. In view of the long-term foreseeable depletion of fossil raw materials, the provision of methods for preparing acrylic esters based on alternative reactants is industrially relevant. An alternative preparation of acrylic acid consists of the reaction of ethylene oxide with carbon monoxide to give poly-3-hydroxypropionate (poly-3HP) and subsequent thermolysis to give acrylic acid.

The preparation of poly-3HP is described, for example, in the dissertation “Multi-Site Catalysis—Novel Strategies to Biodegradable Polyesters from Epoxides/CO and Macrocylic Complexes as Enzyme Models” by Markus Allmendinger, University of Ulm (2003). It is known from this that a product mixture comprising poly-3HP is obtained by carbonylating reaction of ethylene oxide, dissolved in an aprotic solvent, with carbon monoxide at elevated pressure, elevated temperature and in the presence of at least one catalyst system comprising a cobalt source.

J. Am. Chem. Soc. 2002, 124, pages 5646-5647, DE 10137046 A1, WO 03/011941 A2 and J. Org. Chem. 2001, 66, pages 5424-5426 describe further methods for carbonylating reaction of ethylene oxide with carbon monoxide.

WO 2014/012855 A1 describes a method for producing acrylic acid in which ethylene oxide is carbonylated with carbon monoxide in the presence of a cobalt-containing catalyst to give poly-3HP, the cobalt content of the poly-3HP is reduced with water and/or an aqueous solution and the poly-3HP is reacted further to acrylic acid by thermolysis. It was observed that cobalt remaining in the separated poly-3-hydroxypropionate considerably impairs its thermolysis to acrylic acid.

It is desirable to produce acrylic esters from the poly-3HP obtained by reacting ethylene oxide with carbon monoxide without going via acrylic acid. It is known in principle to transesterify poly-3HP to low-molecular weight esters and subsequently to dehydrogenate to produce acrylic esters. The known methods, however, were carried out based on poly-3HP produced by fermentation.

For instance, WO 2013/185009 A1 describes the preparation of alkyl esters of acrylic acid by means of pyrolysis of a genetically modified biomass, which comprises poly-3HP, in the presence of a heat transfer liquid and optionally a catalyst.

WO 03/051813 A1 describes the preparation of intermediates, for example alkyl esters of hydroxycarboxylic acids, from polyhydroxycarboxylates. The polyhydroxycarboxylate isolated preferably from biomass is in this case transesterified by means of an aprotic catalyst in the presence of an alcohol.

The dehydration of 3-hydroxycarboxylic acids is known, for example from US 2005/0222458 A1. This describes a method for dehydrating 3-hydroxycarboxylic acids and polymers thereof in the presence of aluminosilicate esters, optionally with simultaneous esterification, in order to obtain α,β-unsaturated carboxylic acids and esters.

The object of the present invention is to provide a method which allows the preparation of poly-3-hydroxypropionate from ethylene oxide and carbon monoxide and the further conversion to 3-hydroxypropionic acid esters in high yields and with high efficiency.

The object is achieved by a method for preparing a 3-hydroxypropionic acid ester, comprising the following steps:
a) the reaction of ethylene oxide with carbon monoxide in the presence of a cobalt catalyst, wherein poly-3-hydroxypropionate is obtained;
b) the transesterification of the poly-3-hydroxypropionate with an alcohol in the presence of a transesterification catalyst, that is to say a depolymerizing transesterification, wherein the 3-hydroxypropionic acid ester is obtained;
   wherein the transesterification catalyst is a compound of the formula

MLₓ (I)

where
M is a metal of main groups 2, 3 or 4 or transition groups 3 to 9 of the Periodic Table of the Elements, as of 2015,
L is a ligand, which is bonded directly to M via a C, an O, a P, an S and/or an N atom, and
x is an integer from 2 to 6.

The reaction of ethylene oxide with carbon monoxide according to step a) is catalyzed by a cobalt catalyst. The poly-3-hydroxypropionate thus formed includes tough residues of the cobalt catalyst which cannot be removed completely, even by decobalting processes. The methods described in the literature for transesterifying poly-3HP do not make any statement about the influence of cobalt on the transesterification. Since the effect of a catalyst depends on many factors such as, for example, the presence of heavy metals in the compound to be reacted, the influence of cobalt on the transesterification is not predictable. Surprisingly it has now been found that cobalt does not disrupt the transesterification with certain catalysts according to step b), but rather produces even higher yields in combination with the transesterification catalyst and, as the central atom of metal compounds, exhibits its own catalytic effect.

Since polymeric poly-3HP cannot be distilled, complete removal of cobalt as far as possible from poly-3-hydroxypropionate, obtained by cobalt-catalyzed reaction of ethylene oxide with carbon monoxide, is only possible with a high degree of effort. In contrast, the 3-hydroxypriopionic acid esters obtained in the transesterification of poly-3HP according to the invention are generally easier to separate from catalyst residues since they may be isolated by distillation. Since cobalt residues have no disruptive effect during the transesterification and, together with residues of the transesterification catalyst, can be removed from the 3-hydroxypropionic acid esters by distillation, they do not have to be removed from the poly-3HP to the same extent for the method according to the invention as would be advantageous for a thermolysis. The method according to the invention enables the preparation of 3-hydroxypropionic acid esters from carbon monoxide and ethylene oxide in high yields and with high purity.

Poly-3-hydroxypropionate is obtained in the reaction of ethylene oxide with carbon monoxide. The term poly-3-hydroxypropionate (poly-3HP) is understood to mean polyesters of the structure

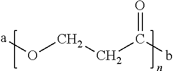

n is an integer ≥2, and may be up to 150 for example, or up to 200, or up to 500 and more.
a, b form the end groups terminating the polyester, the nature of which is dependent on the production conditions (e.g. on the catalyst system used).

For example,

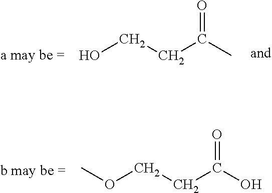

Alternatively,

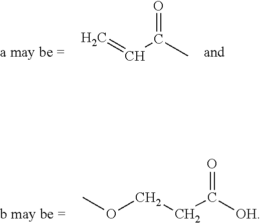

Alternatively,

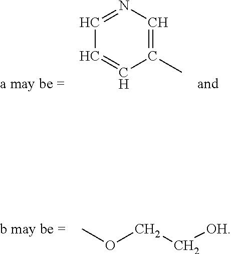

Alternatively,

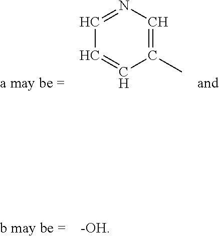

Ethylene oxide is reacted with carbon monoxide in the presence of a cobalt catalyst. A cobalt catalyst is understood to mean a catalyst system comprising at least one cobalt source.

Based on the molar amount of ethylene oxide, the molar amount of cobalt present in the at least one cobalt source of the catalyst system is normally in the range from 0.005 to 20 mol %, preferably in the range from 0.05 to 10 mol %, particularly preferably in the range from 0.1 to 8 mol % and especially preferably in the range from 0.5 to 5 mol %.

It is possible to use any chemical compound comprising cobalt as a suitable cobalt source since this is in each case generally converted into the actual catalytically active cobalt compound under the carbon monoxide pressure to be applied in the method.

Suitable cobalt sources include, inter alia, e.g. cobalt salts such as cobalt chloride, cobalt formate, cobalt acetate, cobalt acetylacetonate, cobalt sulfate and cobalt 2-ethylhexanoate (“cobalt soap”), which are readily carbonylated under the carbon monoxide pressures to be applied (“in situ”; the presence of low amounts of molecular hydrogen may have an advantageous impact in this regard). It is also possible to use finely divided cobalt metal (e.g. in the form of dust) as cobalt source in the method.

Preferred cobalt sources used are already pre-formed cobalt carbonyl compounds (understood hereinafter to mean compounds comprising at least one cobalt atom and at least one carbon monoxide ligand), among which dicobalt octacarbonyl (Co₂(CO)₈) is especially preferred (this comprises [Co(CO)₄]⁻ effectively pre-formed as [Co(CO)₄]⁺[Co(CO)₄]⁻. In an appropriate manner from an application point of view, it is used as the sole cobalt source of the catalyst system.

Especially advantageous is the use of co-catalysts (at least one) in the catalyst system to be used comprising at least one cobalt source, which have at least one nucleophilic Brønsted basic functionality and at least one Brønsted acidic functionality.

These co-catalysts include, in particular, aromatic nitrogen heterocycles (these may be, for example, 5-, 6- or 7-membered rings; they have at least one nitrogen atom in the aromatic ring (cycle)), which have covalently bonded in the molecule, in addition to the Brønsted basic nitrogen, at least one Brønsted acidic (free) hydroxyl group (—OH) and/or at least one Brønsted acidic (free) carboxyl group (—COOH). The aromatic nitrogen heterocycle may in turn be fused with other aromatic and/or aliphatic (e.g. 5-, 6- or 7-membered) ring systems. The at least one hydroxyl group and/or carboxyl group may be located either on the aromatic base nitrogen heterocycle (preferably) or (and/or) on the fused aliphatic and/or aromatic ring system. Obviously, the fused portion may also have one or more than one nitrogen atom as heteroatom. Besides the at least one hydroxyl group and/or carboxyl group, for example aliphatic, aromatic and/or halogen substituents may also be additionally present.

Particularly preferred co-catalysts include, for example, 2-hydroxypyridine, 3-hydroxypyridine, 4-hydroxypyridine, 3,4-dihydroxypyridine, 3-hydroxyquinoline, 4-hydroxy-2-methylpyridine, 3-hydroxy-4-methylpyridine, 2,6-dihydroxypyridine, 2-hydroxyquinoline, 1-hydroxyisoquinoline, 3-hydroxyquinoline, 2,3-dihydroxyquinoxaline, 8-hydroxyquinoline, 2-pyridylmethanol, 3-pyridylmethanol and 2-(2-pyridyl)ethanol. Obviously, as already stated, a carboxyl group may be present instead of or in addition to the hydroxyl group, as is the case with nicotinic acid. Very particular preference is given to using 3-hydroxypyridine as co-catalyst in the preparation method (this particularly in combination with diglyme as aprotic solvent and dicobalt octacarbonyl as cobalt source of the catalyst system).

Generally, the co-catalyst in the reaction is used in total molar amounts M_{Co-cat} such that the ratio M_{Co-cat}:M_{Cobalt}, formed with the total molar amount M_{Cobalt} of cobalt present in the catalyst system used, is from 5:1 to 1:5, preferably from 4:1 to 1:4, particularly preferably from 3:1 to 1:3 and especially preferably from 2:1 to 1:2 or from 2:1 to 1:1.

Carbon monoxide is preferably used in excess (relative to the reaction stoichiometry) in all cases above.

For a reaction of this kind, it is also possible to use salts of the anion [Co(CO)₄]⁻ and/or its Brønsted acid HCo(CO)₄ as cobalt sources. Examples of such salts are tetramethylammonium tetracarbonylcobaltate(-I), Et₄NCo(CO)₄, and bis(triphenylphosphoranylidene)ammonium tetracarbonylcobaltate(-I). For example, DE 10149269 A1 discloses further examples.

Ethylene oxide is reacted with carbon monoxide preferably in a solvent. The type and amount of solvent are preferably selected such that they are sufficient under the reaction conditions to be used to keep the required amount of catalyst system comprising the cobalt in solution in the reaction mixture since the method is preferably carried out with homogeneous catalysis.

A solvent is understood to mean a solvent for poly-3HP. Poly-3HP is preferably soluble in the solvent at 25° C. to an extent of at least 25 g (poly-3HP)/100 g (solvent), particularly preferably to an extent of at least 40 g (poly-3HP)/100 g (solvent). The solvent preferably has a boiling point of more than 20° C. The solubility of poly-3HP in the solvent is dependent on the molecular weight of the poly-3HP. The suitability of a solvent as solvent in the reaction is dependent on the solubility of the poly-3HP produced in the reaction.

In a preferred embodiment, the reaction of ethylene oxide with carbon monoxide takes place in an aprotic solvent. An aprotic solvent is understood to mean organic compounds (and also mixtures of two or more than two of such compounds) which do not comprise any atom other than carbon (any atom type other than carbon) to which a hydrogen atom is covalently bonded, and which are neither ethylenically nor alkynically (in each case mono- or poly)unsaturated. The solvent at a pressure of 1.0133·10⁵ Pa (=standard pressure) is liquid at at least one of the temperatures in the range from 0° C. to 50° C., preferably at at least one of the temperatures in the range from 5° C. to 40° C. and particularly preferably at at least one of the temperatures in the range from 10° C. to 30° C.

Suitable aprotic solvents are those which comprise at least one covalently bound oxygen atom, preferably an ether oxygen atom, i.e. an oxygen atom which forms an ether bridge.

It is furthermore advantageous if the aprotic solvent is or comprises a substance which comprises as atom types other than carbon and hydrogen at most oxygen and/or sulfur.

Suitable aprotic solvents are those whose relative static permittivity ε (also referred to as dielectric constant, dielectric number or permittivity number) at a temperature of 293.15K and a pressure of 1.0133·10⁵ Pa (=standard pressure) as pure liquid substance is in the range from 2 to 50, preferably 3 to 20, particularly preferably 4 to 15 and especially preferably 5 to 10 (the relative static permittivity ε of a vacuum=1).

If at 293.15K and standard pressure the aprotic substance is not liquid but solid, the figure above refers to the temperature of its melting point at standard pressure. If at 293.15K and standard pressure the aprotic substance (aprotic (chemical) compound) is not liquid but gaseous, the figure above refers to a temperature of 293.15K and the associated saturation vapor pressure (the (intrinsic) vapor pressure at which the substance condenses at 293.15K).

A suitable source having figures for relative static permittivities of suitable relevant aprotic substances is, for example, the HANDBOOK of CHEMISTRY and PHYSICS, 92nd Edition (2010-2011), CRC PRESS. According to the figures therein, the relevant ε is e.g. for tetrahydrofuran=7.56, for ethylene oxide=12.43, for 1,4-dioxane=2.22, for ethylene glycol dimethyl ether (1,2-dimethoxyethane)=7.41, for diethylene glycol dimethyl ether (diglyme)=7.38 and for triethylene glycol dimethyl ether (triglyme)=7.62.

Especially preferred aprotic solvents are therefore those for which ε is 2 to 35, advantageously 3 to 20, particularly advantageously 4 to 15, and especially advantageously 5 to 10, and at the same time comprise at least one covalently bound oxygen which is with particular advantage an ether oxygen atom.

Suitable aprotic solvents for the reaction include for example:

saturated (cyclic and acyclic) and aromatic hydrocarbons such as n-hexane, n-heptane, petroleum ether, cyclohexane, benzene and toluene,
halogenated saturated and aromatic hydrocarbons such as dichloromethane, chlorobenzene and 1,4-dichlorobutane,
esters of organic acids (particularly organic carboxylic acids) such as n-butyl propionate, phenyl acetate, glyceryl triacetate, ethyl acetate, diethyl phthalate and dibutyl phthalate,
ketones such as acetone, ethyl methyl ketone, methyl isobutyl ketone, benzophenone, cyclohexanone and 2,4-dimethyl-3-pentanone,
nitriles such as acetonitrile, propionitrile, n-butyronitrile and benzonitrile,
dialkylamides such as dimethylformamide and dimethylacetamide,
carbonic esters such as dimethyl carbonate, diethyl carbonate, ethyl methyl carbonate, ethylene carbonate and propylene carbonate,
sulfoxides such as dimethyl sulfoxide,
sulfones such as sulfolane,
N-alkylpyrrolidones such as N-methylpyrrolidone and
cyclic and acyclic ethers such as diethyl ether, anisole (methyl phenyl ether), tetrahydrofuran, 2-methyltetrahydrofuran, 1,4-dioxane, diphenyl ether, benzyl methyl ether, ethoxybenzene, 1,2-dimethoxybenzene, alkylene glycol dialkyl ether (e.g. ethylene glycol dialkyl ethers such as ethylene glycol dimethyl ether (=monoglyme)) and polyalkylene glycol dialkyl ethers such as diethylene glycol diethyl ether, diethylene glycol dimethyl ether (=diglyme), triethylene glycol dimethyl ether (=triglyme), tetraethylene glycol dimethyl ether (=tetraglyme), dipropylene glycol dimethyl ether (=proglyme) and diethylene glycol dibutyl ether (=butyl digylme).

The solvent is preferably selected from alkylene glycol dialkyl ethers, polyalkylene glycol dialkyl ethers, 1,4-dioxane, methyl phenyl ether, cyclohexanone, 2,4-dimethyl-3-pentanone, chlorobenzene, 1,4-dichlorobutane, diethyl carbonate, ethyl acetate, N-methylpyrrole, ethoxybenzene, 1,2-dimethoxybenzene, tetrahydrofuran, 2-methyltetrahydrofuran, benzyl methyl ether and diethyl phthalate.

Of these, particular preference is given to alkylene glycol dialkyl ethers and polyalkylene glycol dialkyl ethers, such as ethylene glycol dimethyl ether (1,2-dimethoxyethane, monoglyme), diethylene glycol dimethyl ether (bis(2-methoxyethyl) ether; diglyme), dipropylene glycol dimethyl ether (proglyme) and diethylene glycol dibutyl ether (=butyl diglyme). Diglyme is especially preferred.

The reaction temperature and the working pressure (the absolute pressure in the gaseous atmosphere of the reaction chamber) used during the reaction of ethylene oxide with carbon monoxide are not critical and may vary over wide limits. The reaction can be conducted under comparatively mild reaction conditions. Suitable reaction temperatures are in the range from 25 to 150° C., preferably in the range from 35 or 50 to 120° C., particularly preferably in the range from 60 to 100° C. and especially preferably in the range from 70 to 90° C.

At comparatively low temperatures, the reaction proceeds at a somewhat reduced reaction rate but with a comparatively enhanced target product selectivity, which is virtually 100 mol %. By means of superatmospheric working pressures, the reaction according to the invention is favored. Appropriately, from an application point of view, the working pressure in the reaction normally does not exceed 2.5·10⁷ Pa, since higher working pressures can result in excessive plant costs. Working pressures from 2·10⁵ Pa to 2·10⁷ Pa are advantageous in accordance with the invention. A working pressure applied in the reaction is preferably in the range from 5·10⁵ Pa to 1.5·10⁷ Pa, particularly preferably in the range from 1·10⁶ Pa to 1·10⁷ Pa and especially preferably in the range from 2·10⁶ Pa to 12·10⁶ Pa or in the range from 4·10⁶ Pa to 10·10⁶ Pa. The reaction is typically carried out in a positive pressure reactor such as an autoclave.

Oxidizing gases (e.g. O₂, N₂O), CO₂ and water normally act as catalyst poisons with respect to the reaction according to the invention or react with ethylene oxide to give by-products and are therefore largely or preferably completely excluded from the carbon monoxide to be used (or generally from the components of the reaction mixture to be used). Their individual proportions by volume to the total volume of carbon monoxide used should be ≤1% by volume, better ≤0.1% by volume, preferably ≤0.01% by volume, particularly preferably ≤0.001% by volume and especially preferably are insignificant.

Accordingly, the reaction according to the invention is carried out preferably under inert conditions, i.e. in the absence of moisture and air. Since ethylene oxide forms a highly flammable gas, the presence of molecular oxygen in the carbonylation according to the invention is also disadvantageous from this point of view. Water is also capable of opening the ethylene oxide ring in an undesirable manner, which is why the presence of water, as steam for example, (apart from the low amounts already mentioned) in the positive pressure vessel is also undesirable from this perspective.

The carbon monoxide to be used for the reaction according to the invention can therefore be fed to the positive pressure reactor either in a mixture with inert gases (e.g. N₂, noble gases such as Ar) or essentially as a pure substance. The latter is preferred, which is why the working pressures listed above for the reaction also form favorable (in the gaseous atmosphere of the reaction chamber) CO partial pressures for the carbonylation according to the invention.

The reaction according to the invention is normally carried out in a gas-tight sealable pressure vessel for reactions under positive pressure, for example in an autoclave. In principle, the poly-3HP formation can be carried out in a positive pressure reactor both in batchwise mode and continuously. If it is carried out in batchwise mode, the working pressure (and with this the CO partial pressure) can be kept constant, or the reaction of the carbonylation may subsequently decrease. The former is possible in a simple fashion by always repressurizing the consumed CO in the reaction chamber of the pressure reactor.

Carbon monoxide with a purity of 99% by volume or higher, especially 99.5% by volume or higher, is suitable for a reaction according to the invention.

Furthermore, ethylene oxide with a purity of 99.9% by weight or higher can be used as raw material for the reaction according to the invention (the figures refer to the liquid phase). In this case, a residual aldehyde content of the ethylene oxide can be removed before use in a manner known per se by treating the same with aldehyde scavengers (e.g. aminoguanidine hydrogencarbonate).

Normally, in the case of a batchwise reaction according to the invention, it is carried out such that the reaction chamber of the autoclave is initially flushed with inert gas (e.g. Ar) in an appropriate manner in application terms. Subsequently, under an inert gas atmosphere and at comparatively low temperature, the catalyst system, the aprotic solvent and the ethylene oxide is placed in the reaction chamber of the autoclave and the autoclave is sealed.

The reaction chamber is preferably operated with stirring. By means of an appropriate pressure valve, a suitable amount of carbon monoxide for the purpose of the carbonylation is then compressed into the reaction chamber of the autoclave.

The temperature in the reaction chamber is then increased to the reaction temperature by external heating and the reaction mixture is stirred in the autoclave, for example while maintaining the reaction temperature. If no further carbon monoxide is compressed into the reaction chamber during the course of the reaction, the reaction is then generally terminated when the internal pressure in the reaction chamber falls to a value unchanging over time. The temperature in the interior of the reaction chamber is lowered by appropriate cooling, the elevated internal pressure is subsequently depressurized to atmospheric pressure and the autoclave is opened so that access is provided to the product mixture in the reaction chamber itself. As already mentioned, the carbon monoxide is normally used in superstoichiometric amounts, particularly when performing the reaction according to the invention in batchwise mode. In principle, however, the corresponding stoichiometric amount of CO can also be used in the method according to the invention.

Based on the total molar amount of ethylene oxide fed into the autoclave and in comparatively short reaction times (generally 0.5 to 3.0 h), typically conversions of ≥80 mol %, advantageously ≥90 mol % or 95 mol %, preferably ≥98 mol %, and particularly preferably ≥99 mol % can be achieved.

In the reaction of ethylene oxide with carbon monoxide, a product mixture is formed which generally comprises at least the main amount of the poly-3HP formed in the dissolved state.

The poly-3HP concentration in the solution is preferably in the range from 5 to 35% by weight, particularly 10 to 20% by weight. In addition to the solvent and the dissolved poly-3HP, the solution may comprise further constituents, for example (co)catalysts and by-products of the preceding poly-3HP synthesis. Solvent and dissolved poly-3HP together make up preferably at least 80% by weight, particularly preferably at least 90% by weight and especially preferably at least 98% by weight of the solution.

The poly-3HP can be isolated from the reaction mixture by precipitation with subsequent solid-liquid separation or by forming a liquid poly-3HP phase followed by liquid-liquid separation.

In one embodiment, poly-3-hydroxypropionate is precipitated from the solution of poly-3HP in the aprotic solvent by adding an antisolvent. A solution obtained in the reaction is preferably used as such in the precipitation process, i.e. without intermediate work-up,

For the purposes of the present application, an antisolvent is understood to mean a solvent in which the poly-3HP is soluble at 25° C. to an extent of at most 1 g (poly-3HP)/100 g (antisolvent) and which has a boiling point of more than 20° C. The solubility of poly-3HP in the solvent is dependent on the molecular weight of the poly-3HP. The suitability of a solvent as antisolvent in the precipitation is dependent on the solubility of the poly-3HP produced in the reaction. For undisrupted implementation of the precipitation process, the solvent and the antisolvent are preferably at least partially miscible with each other and are in particular fully miscible at the weight ratio of solution to antisolvent used.

Alcohols are less preferred as antisolvent. For example, if methanol is used as antisolvent, esters are formed with terminal carboxyl groups of the poly-3HP, which in the case of subsequent further use can lead to the formation of undesired by-products, for example methyl acrylate.

The antisolvent is preferably water or an aqueous solution. The reaction of ethylene oxide with carbon monoxide is particularly preferably carried out in an aprotic solvent, and poly-3-hydroxypropionate is precipitated from the resulting solution of poly-3-hydroxypropionate in the aprotic solvent by adding an aqueous antisolvent.

The pH of the aqueous solution used as antisolvent, at a temperature of 25° C. and at standard pressure, is generally ≤7.5, advantageously ≤7. The aforementioned pH of the aqueous antisolvent is preferably ≤6, and particularly preferably ≤5, and especially preferably ≤4. In general, the aforementioned pH of the aqueous antisolvent does not go below 0 and is frequently ≥1 or ≥2.

The aforementioned pH values (likewise based on 25° C. and standard pressure) advantageously also apply to the resulting aqueous mixtures on adding the aqueous antisolvent to the poly-3HP solution, which are optionally advantageously treated with a gas comprising molecular oxygen, and from which the precipitated poly-3HP is separated off by using at least one mechanical separation operation. The pH (25° C., standard pressure) of these aqueous mixtures is preferably from 2 to 4, for example 3.

To set the relevant pH, inorganic and/or organic acids (in the sense of Brønsted) are suitable. Examples of inorganic acids include sulfuric acid, carbonic acid, hydrochloric acid and/or phosphoric acid. Preference is given to using organic carboxylic acids, especially alkanoic acids, as pH modifiers. Listed among these are, for example, acrylic acid, oxalic acid, formic acid, acetic acid, propionic acid, fumaric acid and/or maleic acid. To adjust the relevant pH, it will be appreciated that organic sulfonic acids such as methanesulfonic acid can also be used or incorporated.

Suitable aqueous antisolvents therefore, for example, are aqueous solutions comprising one or more than one of the dissolved aforementioned inorganic and/or organic acids. Aqueous antisolvents of this kind are, for example, aqueous sulfuric acid, aqueous carbonic acid, aqueous hydrochloric acid, aqueous phosphoric acid, aqueous acrylic acid, aqueous oxalic acid, aqueous formic acid, aqueous acetic acid, aqueous propionic acid, aqueous fumaric acid, aqueous maleic acid and/or aqueous methanesulfonic acid. It will be appreciated that the addition of water and one or more acids for the purposes of precipitation of poly-3HP may also be carried out temporally and/or spatially separately from each other such that the acidic aqueous antisolvent effectively added only forms for example in the aqueous mixture comprising the poly-3HP.

An advantage of the use of carboxylic acids is the enhanced solubility of the catalyst, oxidized to cobalt cations in the subsequent step, through the formation of salts, soluble in the reaction solution, consisting of cobalt cations and carboxylic acid anions. Acetic acid, acrylic acid and propionic acid are particularly suitable.

Appropriately from an application point of view, one of the aforementioned suitable aqueous antisolvents comprises, based on the weight of the aqueous liquid, at least 10% by weight, better at least 20% by weight or at least 30% by weight, advantageously at least 40% by weight or at least 50% by weight, particularly advantageously at least 60% by weight or at least 70% by weight, optionally at least 80% by weight or at least 90% by weight, frequently at least 95% by weight, or at least 97% by weight, or at least 99% by weight water.

Advantageously, the carboxylic acid is selected from acetic acid and propionic acid and especially preferably the aqueous solution comprises 5% by weight to 30% by weight, particularly preferably 7% by weight to 25% by weight carboxylic acid, especially preferably 10 to 15% by weight carboxylic acid.

The antisolvent is preferably added to the solution that has a temperature of 10 to 90° C. The term temperature here is understood to mean the temperature of the mixture of antisolvent and solution. The mixture has a temperature of 10 to 90° C. over the entire period of the antisolvent addition.

The weight ratio of solution to antisolvent (m_{L}/m_{A}) is preferably from 0.3 to 3, especially 0.5 to 1. In the case of higher values of m_{L}/m_{A}, no precipitation is achieved since the amount of antisolvent is too low to produce a sufficient supersaturation of the solution of poly-3HP. At lower values of m_{L}/m_{A}, greater amounts of liquid have to be separated from the solid which leads to a longer filtration time.

When feeding the gas comprising molecular oxygen, a gas dispersion is formed at sufficiently high stirring power, which allows a large contact area of the gas comprising oxygen with the solution or suspension and thus advantageously influences the oxidation of the cobalt catalyst. The stirring energy power input is preferably at least 0.3 W per kg of the total mass of solution and antisolvent. At lower power input, the gas dispersion does not mix to a sufficient degree or separates out, which adversely affects the decobalting. The power input is particularly preferably at least 0.5 W per kg of the total mass of solution and antisolvent. The stirring power is particularly preferably in the range from 0.8 to 2.0 W per kg of the total mass of solution and antisolvent.

The addition of the antisolvent and the feeding of the gas comprising molecular oxygen are preferably carried out in parallel or overlapping or separately over time.

The antisolvent is added while mixing, e.g. while stirring. The stirring energy power input is preferably controlled. The power input from the start of the antisolvent addition up to the separation of the precipitated poly-3HP is preferably 0.1 to 10 W per kg of the total mass of solution and antisolvent, particularly preferably 0.3 to 3 W per kg of the total mass of solution and antisolvent. At a relatively low stirring energy power input, there is insufficient extraction of the cobalt from the poly-3HP particles.

During and/or after addition of the antisolvent, a gas comprising molecular oxygen is preferably fed to the solution. Advantageously, the gas comprising molecular oxygen is air or comprises air. The supply of gas comprising molecular oxygen serves to oxidize the cobalt catalyst. The salts of the cobalt cations originating from the oxidation have different degrees of solubility in water depending on the counterion(s). The anions of the carboxylic acids preferably used, particularly acetate and propionate, form readily soluble salts with cobalt cations in the reaction solution.

It is preferable, for example, to carry out the aforementioned addition in the presence of air and/or in the presence of a gas other than air comprising molecular oxygen. This may be accomplished in a simple manner by passing through the intensively comixed agueous mixture of molecular oxygen or gas comprising molecular oxygen.

The gas comprising molecular oxygen is preferably fed to the solution or suspension during and/or after the precipitation at a temperature of e.g. 10 to 90° C., or 20 to 90° C., or 30 to 90° C., advantageously 40 to 90° C. The term temperature here refers to the temperature of the solution or suspension over the entire period of the feeding of the gas comprising molecular oxygen. If the solvent is an alkylene glycol dialkyl ether such as diglyme, the temperature is preferably 40 to 50° C.; if the solvent is an ester of an organic acid sich as diethyl phthalate, the temperature is preferably 65 to 90° C.

Subsequently, the aqueous mixture can be cooled to temperatures of ≤25° C., preferably ≤20° C. and particularly preferably ≤15° C. or ≤10° C., in order to promote the precipitation of the poly-3HP.

Finally, the precipitated poly-3HP is separated off from the liquid mixture by at least one solid-liquid separation step. It will be appreciated that the separation of precipitated poly-3HP may also be carried out while the liquid mixture is still warm.

The liquid phase remaining in this case (which comprises a further portion of the product mixture) can be further processed (e.g. for the purpose of increasing the yield of poly-3HP separated off) in a corresponding manner (however a sufficient initial amount of antisolvent to be added can already in principle be selected such that the desired target amount of poly-3HP already precipitates in the first precipitation step).

The poly-3HP separated off is typically subjected to a wash step. The wash step is preferably carried out preferably with deionized water, wherein preferably a ratio of wash agent mass (WA mass) to suspension mass (susp. mass) is selected in the range from 0.05 to 5.0, particularly preferably from 0.07 to 3.0. The washing is carried out as described in the examples. Alternatively, a macerating wash is also possible after which it is filtered again.

An alternative to the precipitation of the poly-3HP by adding an antisolvent and subsequent solid-liquid separation is the formation of a poly-3HP liquid phase and subsequent liquid-liquid separation. For liquid-liquid separation, an antisolvent is added to the mixture obtained from the reaction of ethylene oxide with carbon monoxide which is essentially immiscible with the solvent and triggers the formation of a phase comprising poly-3HP. In the poly-3HP phase, the poly-3HP is present in the solvent in dissolved form and/or is present as a melt at elevated temperature. Suitable solvents, which are immiscible with an aqueous antisolvent, preferably aqueous acetic acid, aqueous propionic acid or aqueous acrylic acid, particularly include butyl diglyme, anisole, chlorobenzene, diethyl phthalate, 1-methylpyrrole, 1,4-dichlorobutane and diethyl carbonate. The poly-3HP phase can be separated off by liquid-liquid separation. Cobalt salts, which are formed during work-up of the reaction mixture from a), largely migrate into the antisolvent and can therefore be separated from the poly-3HP, wherein residues of the cobalt catalyst always remain in the poly-3HP. During and/or after addition of the antisolvent, a gas comprising molecular oxygen is preferably fed to the mixture, as described above.

It has been shown that residues of the cobalt catalyst present in the poly-3-hydroxypropionate do not impair the transesterification, but rather produce even higher yields. A cobalt concentration in the poly-3-hydroxypropionate that is too low is therefore undesirable. The poly-3HP used in the transesterification according to the invention preferably has cobalt contents in the range from 0.01 to 5.0% by weight, particularly preferably in the range from 0.01 to 2.0% by weight, especially preferably from 0.01 to 0.7% by weight.

The poly-3-hydroxypropionate is transesterified with an alcohol in the presence of a transesterification catalyst. The transesterification catalyst used is a compound of the formula

MLₓ (I).

Here, M is a metal of main groups 2, 3 or 4 or transition groups 3 to 9 of the Periodic Table of the Elements, L is a ligand which is bonded directly to M via a C, an O, a P, an S and/or an N atom, and x is an integer from 2 to 6, preferably 2 to 4, particularly preferably 2 to 3. Especially preferably, x corresponds to the oxidation state of the metal M.

Based on the amount of poly-3HP, the amount of transesterification catalyst used is normally in the range from 0.001 to 10% by weight, preferably in the range from 0.005 to 5% by weight, particularly preferably in the range from 0.01 to 3% by weight and especially preferably in the range from 0.01 to 1.5% by weight.

In other words, the amount of transesterification catalyst used, based on the amount of ethylene oxide in the reaction a), is normally in the range from 0.001 to 10 mol %, preferably in the range from 0.005 to 5 mol %, particularly preferably in the range from 0.01 to 3 mol % and especially preferably in the range from 0.1 to 1.5 mol %.

The central atom M is preferably a metal from main group 3 or 4 or transition group 4 or 9 of the Periodic Table of the Elements. M is particularly preferably titanium, aluminum or cobalt.

Preference is given to combinations of a catalyst in which M is cobalt, and a catalyst in which M is a metal from main group 2, 3 or 4 or transition groups 3 to 9 of the Periodic Table of the Elements. Particular preference is given to combinations of a catalyst in which M is cobalt, and a catalyst in which M is a metal from main group 3 or 4 or of transition group 4 of the Periodic Table of the Elements, especially titanium or aluminum. In the catalyst in which M is cobalt, there are preferably traces of cobalt salts, especially Co(II) acetate, which form during the work-up of the reaction mixture from a) and remain in the poly-3HP during the decobalting.

The ligand L can be either a chelating ligand or a non-chelating ligand. Preference is given to anionic ligands. Ligands L can be the same or different from one another. The compound of the formula (I) preferably comprises exclusively non-chelating ligands.

If the compound of the formula (I) comprises one or more chelating ligands, the at least two atoms binding to the central atom of the chelate ligand can be identical, but it is also possible that the atoms binding to the central atom are different atoms. An example of a chelate ligand, in which the atoms binding to the central atom are identical, is acetylacetonate.

If the compound of the formula (I) comprises one or more non-chelating ligands, it is preferable that L is selected from alkyl, alkoxy, alkylcarboxyl, alkylsulfoxy and/or aryl radicals. In such a case it is preferable that the ligand(s) are C₁-C₂₂-alkyl, C₁-C₂₂-alkoxy, C₁-C₂₂-alkylcarboxyl, C₁-C₂₂-alkylsulfoxy or C₆-C₂₂-aryl radicals. Preference is given to C₁-C₈-alkyl, C₁-C₈-alkoxy, C₁-C₈-alkylcarboxyl or C₆-C₈-aryl radicals.

Examples of suitable compound classes which can be used in accordance with the invention as transesterification catalyst are metal halides, metal acid esters, metal organyls, organometallic alkoxides, organometallic halides, organometallic hydrides, organometallic carboxylates, organometallic amides, organometallic sulfinates, organometallic sulfonates and metal complexes of the metallocene type.

An example of a suitable compound of the formula (I) with a metal of main group 2 of the Periodic Table of the Elements is magnesium(II) mesylate.

Examples of suitable compounds of the formula (I) with a metal from main group 3 of the Periodic Table of the Elements are aluminum chloride, triethylaluminum, triisobutylaluminum, aluminum tri(isopropoxide) (Al(III) isopropoxide), aluminum triacetate (Al(III) acetate), aluminum tri(hydroxyacetate) (Al(III) hydroxyacetate), diethylaluminum chloride, diethylaluminum hydride, diethoxy(methyl)aluminum, ethoxy(dimethyl)aluminum, triethoxyaluminum, aluminum tri(ethylacetoacetonate), aluminum trimesylate (Al(III) mesylate), gallium (acetylacetonate), indium triacetate and indium tris(isopropoxide). Preference is given to Al(III) mesylate, Al(III) isopropoxide, Al(III) acetate and Al(III) hydroxyacetate, especially Al(III) mesylate.

Examples of suitable compounds of the formula (I) with a metal from main group 4 of the Periodic Table of the Elements include germanium (isopropoxide), triethyltin ethoxide, tin(II) chloride, tin(II) octoate, tin(II) ethylhexanoate, tin(II) laurate, dibutyltin oxide, dibutyltin dichloride, dibutyltin diacetate, dibutyltin dilaurate, dibutyltin maleate, dioctyltin diacetate, tin(II) 2-ethylcaproate, tin(II) ethylhexanoate, triethyltin dimethylamide, cyclopentadienyltrimethyltin, bis(cyclopentadienyl)tin and lead acetate.

Examples of suitable compounds of the formula (I) with a metal from transition group 3 of the Periodic Table of the Elements are scandium (isopropoxide) and scandium(III) trifluoromethanesulfonate.

Examples of suitable compounds of the formula (I) with a metal from transition group 4 of the Periodic Table of the Elements include titanium tetrachloride, tetraethyl orthotitanate, tetraisopropyl titanate, tetrabutyl titanate (Ti(IV) butoxide), tetramesyl titanate (Ti(IV) mesylate), chlorotriisopropyl orthotitanate, tetra(phenylmethylene)titanium, bis(cyclopentadienyl)titanium dichloride and titanium acetylacetonate. Preference is given to Ti(IV) mesylate and Ti(IV) butoxide.

Examples of suitable compounds of the formula (I) with a metal from transition group 5 of the Periodic Table of the Elements include vanadium(V) oxytriisopropoxide, vanadium(III) acetylacetonate and niobium(V) ethoxide.

Examples of suitable compounds of the formula (I) with a metal from transition group 6 of the Periodic Table of the Elements include chromium trichloride, chromium(III) acetylacetonate, molybdenum pentachloride and molybdenum glycolate.

Examples of suitable compounds of the formula (I) with a metal from transition group 7 of the Periodic Table of the Elements include manganese dichloride, manganese acetate and rhenium trichloride.

Examples of suitable compounds of the formula (I) with a metal from transition group 8 of the Periodic Table of the Elements include iron trichloride, iron tribromide, iron(III) octoate, iron(III) acetylacetonate, iron(III) citrate and iron(II) gluconate.

Examples of suitable compounds of the formula (I) with a metal from transition group 9 of the Periodic Table of the Elements include the cobalt salts mentioned above, especially Co(II) acetate, cobalt(II) acetylacetonate and cobalt(III) acetylacetonate. Preference is given to Co(II) acetate.

The transesterification catalyst is preferably selected from Ti(IV) mesylate, Ti(IV) butoxide, Al(III) mesylate, Al(III) isopropoxide, Al(III) acetate, Al(III) hydroxyacetate and Co(II) acetate. The transesterification catalyst is particularly preferably selected from Ti(IV) butoxide, Ti(IV) mesylate and Al(III) mesylate.

In one of the embodiments, a compound selected from the co-catalysts described above is used concomitantly in analogous amounts in the transesterification.

The poly-3-hydroxypropionate is transesterified with an alcohol. The alcohol is preferably a C₁-C₁₈-alcohol, particularly preferably a C₁-C₈-alcohol. The alcohol can be saturated or unsaturated, substituted or unsubstituted, branched or straight-chain, cyclic or acyclic. The alcohol can be a monohydric or polyhydric alcohol.

Examples of suitable alcohols include methanol, ethanol, propanol, butanol, 2-ethylhexanol, cyclohexanol, decyl alcohol, dodecyl alcohol, isopropyl alcohol, isobutyl alcohol, dodecyl alcohol, stearyl alcohol, oleyl alcohol, linoleyl alcohol, linolenyl alcohol, propylene glycol, glycerol, ethylene glycol, propylene glycol, 1,2-propanediol, 1,3-propanediol, 1,3-butanediol, 1,4-butanediol, 1,6-hexanediol, glycerol, erythritol, pentaerythritol, dipentaerythritol, trimethylolpropane, xylose, sucrose, dextrose and triethanolamine. Particular preference is given to methanol, ethanol, cyclohexanol, butanol and 2-ethylhexanol.

The weight ratio of alcohol to poly-3HP in the esterification mixture is advantageously at least 1:1, preferably at least 2:1, especially preferably at least 5:1, most preferably at least 10:1.

The transesterification can be carried out in a solvent. In one embodiment, the transesterification is carried out in an aprotic solvent specified for the reaction of ethylene oxide and carbon monoxide.

In a preferred embodiment, the alcohol serves as reactive solvent. The esterification mixture particularly preferably does not comprise any further solvent in addition to the alcohol.

Poly-3-hydroxypropionate is typically transesterified with the alcohol at elevated temperature and/or elevated pressure to give a 3-hydroxypropionic acid ester. It should be noted that the reaction conditions must be selected such that the activation energy of the respective transesterification is provided. For example, for the transesterification of poly-3-hydroxypropionate with n-butanol at a pressure of 1 bar, a temperature of at least 125° C. is required. Transesterifications with alcohols such as n-butanol, the boiling point of which is below the required temperature, are preferably conducted at elevated pressure.

The temperature is typically at least 80° C., preferably at least 110° C., particularly preferably at least 120° C. and especially preferably at least 130° C. The temperature is typically at most 220° C., preferably at most 210° C., particularly preferably at most 200° C. The temperature is, for example, 130 to 200° C., preferably 150 to 190° C., particularly preferably 160 to 180° C.

The pressure is typically at least 1 bar, preferably at least 3 bar, particularly preferably at least 5 bar. The pressure is typically at most 35 bar, preferably at most 20 bar. The pressure is, for example, 1 bar to 20 bar, preferably 1 bar to 15 bar, particularly preferably 1 bar to 10 bar. The pressure is typically adjusted by metered addition of inert gas, for example nitrogen, helium, argon, krypton or xenon.

The reaction vessel suitable for the transesterification is selected according to the prevailing pressure. At low pressures for example, a glass flask can be used. In order to achieve as complete a conversion as possible, the water produced during the transesterification can be removed continuously during the reaction. For this purpose a water separator is suitable for example. At elevated pressures, the reaction can be conducted in a gas-tight sealable pressure vessel for reactions under positive pressure, for example in an autoclave. This typically consists of stainless steel.

Oxidizing gases (e.g. O₂, N₂O), CO₂ and water normally act as catalyst poisons with respect to the transesterification according to the invention and/or may cause the formation of by-products and are therefore largely or preferably completely excluded in the transesterification (or generally from the components of the reaction mixture to be used). Their individual proportions by volume to the total volume of carbon monoxide used should be ≤1% by volume, better ≤0.1% by volume, preferably ≤0.01% by volume, particularly preferably ≤0.001% by volume and especially preferably are insignificant.

Normally, in the case of a batchwise transesterification according to the invention, it is carried out such that the reaction chamber of the reaction vessel is initially flushed with inert gas (e.g. Ar) in an appropriate manner in application terms. Subsequently, under an inert gas atmosphere and at comparatively low temperature, the reaction components are placed in the reaction chamber of the reaction vessel and the reaction vessel is sealed. The reaction chamber is preferably operated with stirring.

The temperature in the reaction chamber is then increased to the reaction temperature by external heating and the reaction mixture is fed into the reaction vessel. The reaction temperature of the transesterification typically increases during the course of the reaction due to the formation of high-boiling compounds, namely in the range from 0.5 to 20° C. Samples are advantageously taken at regular intervals from the reaction mixture and the reaction course is monitored by gas chromatography (GC). When the reaction is complete, the temperature in the inner reaction chamber is lowered by appropriate cooling, the elevated internal pressure is subsequently depressurized to atmospheric pressure and the reaction vessel is opened so that access is provided to the product mixture in the reaction chamber itself.

Based on the total molar amount of poly-3HP fed into the autoclave and in comparatively short reaction times (generally 0.3 to 36 h), typically conversions of ≥90 mol %, advantageously ≥95 mol % or ≥98 mol %, preferably ≥99 mol %, and particularly preferably ≥99.9 mol % can be achieved.

In the transesterification of poly-3HP with the alcohol, a reaction mixture is formed which comprises, inter alia, catalyst residues and unreacted alcohol in addition to the 3-hydroxypropionic acid ester.

In one embodiment, a gas comprising molecular oxygen is fed to the reaction mixture of the reaction of ethylene oxide with carbon monoxide for the removal of the catalyst residues. Advantageously, the gas comprising molecular oxygen is air or comprises air. The supply of gas comprising molecular oxygen serves to oxidize the transesterification catalyst. The salts of the metal cations originating from the oxidation have different degrees of solubility in the reaction mixture depending on the counterion(s). For instance, cobalt acetates are poorly soluble salts which can be removed by customary solid-liquid separation methods.

It is preferable, for example, to carry out the aforementioned addition in the presence of air and/or in the presence of gas other than air comprising molecular oxygen. This may be accomplished in a simple manner by passing through the intensively comixed mixture of molecular oxygen or gas comprising molecular oxygen.

When feeding the gas comprising molecular oxygen, a gas dispersion is formed at sufficiently high stirring power, which allows a large contact area of the gas comprising oxygen with the solution or suspension and thus advantageously influences the oxidation of the transesterification catalyst. The stirring energy power input is preferably at least 0.3 W per kg of the mass of the reaction mixture. At lower power input, the gas dispersion does not mix to a sufficient degree or separates out, which adversely affects the oxidation.

The power input is particularly preferably at least 0.5 W per kg of the mass of the reaction mixture. The power input is especially preferably in the range from 0.8 to 2.0 W per kg of the mass of the reaction mixture. Further conditions for feeding a gas comprising molecular oxygen are analogous to the conditions specified for the work-up of poly-3HP.

The liquid reaction mixture separated off from oxidized catalyst by solid-liquid separation is purified preferably by distillation at a pressure of at most 100 mbar, preferably at most 10 mbar, particularly preferably at most 0.1 mbar, in order to isolate the 3-hydroxypropionic acid ester.

In a preferred embodiment, the reaction mixture is purified by means of extraction. Water is added to the reaction mixture in a weight ratio m_{reaction mixture}:m_{water} preferably from 1:5 to 5:1, particularly preferably from 1:3 to 3:1, and especially preferably from 1:2 to 2:1. The aqueous phase is preferably extracted with an organic water-insoluble solvent, anisole for example, in a weight ratio m_{aqueous}:mₛₒₗᵥₑₙₜ preferably from 1:5 to 5:1, particularly preferably from 1:3 to 3:1, and especially preferably from 1:2 to 2:1. The 3-hydroxypropionic acid ester obtained in the organic phase is isolated preferably by distillation at a pressure of at most 100 mbar, preferably at most 10 mbar, particularly preferably at most 0.1 mbar.

The invention also provides a method for preparing an acrylic acid ester comprising the following steps:
(i) the preparation of a 3-hydroxypropionic acid ester according to the method described above; and the
(ii) dehydration of the 3-hydroxypropionic acid ester.

The dehydration of the 3-hydroxypropionic acid ester may be carried out in principle in the liquid phase or in the gas phase.

The dehydration of the 3-hydroxypropionic acid ester is carried out in the liquid phase advantageously at a temperature of 120 to 300° C., preferably of 150 to 250° C., more preferably of 170 to 230° C., most preferably of 180 to 220° C. The pressure is not subject to any restrictions. A slightly reduced pressure is advantageous for safety reasons.

The liquid phase preferably comprises a polymerization inhibitor. Suitable polymerization inhibitors are phenothiazine, hydroquinone and/or hydroquinone monomethyl ether. Very particular preference is given to phenothiazine and hydroquinone monomethyl ether. The liquid phase comprises preferably from 0.001% to 5% by weight, more preferably from 0.01% to 2% by weight and most preferably from 0.1% to 1% by weight of the polymerization inhibitor. Advantageously, an oxygen-containing gas is additionally used to inhibit polymerization. Particularly suitable for this purpose are air/nitrogen mixtures having an oxygen content of ca. 6% by volume (lean air).

The liquid phase generally comprises 5 to 95% by weight, preferably from 10 to 90% by weight, more preferably from 20 to 80% by weight and most preferably from 30 to 60% by weight of an inert organic solvent. The boiling point of the inert organic solvent at 1013 mbar is preferably in the range from 200 to 350° C., particularly preferably from 250 to 320° C., especially preferably from 280 to 300° C. The inert organic solvent generally at 23° C. preferably has a solubility in water of preferably less than 5 g per 100 mL of water, particularly preferably less than 1 g per 100 mL of water, especially preferably less than 0.2 g per 100 mL of water.

Suitable inert organic solvents are, for example, dimethyl phthalate, diethyl phthalate, dimethyl isophthalate, diethyl isophthalate, dimethyl terephthalate, diethyl terephthalate, alkanoic acids such as nonanoic acid and decanoic acid, biphenyl and/or diphenyl ether.

The dehydration may be base- or acid-catalyzed. Suitable basic catalysts are high-boiling tertiary amines, such as pentamethyldiethylenetriamine. Suitable acidic catalysts are high-boiling inorganic or organic acids, such as phosphoric acid and dodecylbenzenesulfonic acid. High-boiling here means a boiling point at 1013 mbar of preferably at least 160° C., more preferably at least 180° C., most preferably at least 190° C.

The amount of dehydration catalyst in the liquid phase is preferably from 1 to 60% by weight, more preferably from 2 to 40% by weight, most preferably from 5 to 20% by weight.

The water/acrylic acid mixture formed in the liquid phase dehydration is preferably removed by distillation, more preferably by means of a rectification column. Further information regarding dehydration in the liquid phase can be found, for example, in WO 2015/036218 A1 and WO 2015/036278 A1.

The 3-hydroxypropionic acid ester is dehydrated in the gas phase preferably by heating a solution of the 3-hydroxypropionic acid ester, for example a solution of the 3-hydroxypropionic acid ester in the alcohol used in the transesterification, in which it is heated particularly preferably in the presence of a catalyst. For example butanol is preferably used as solvent for the dehydration of butyl 3-hydroxypropionate in the gas phase.

The dehydration is effected in the gas phase preferably in the temperature range between 230 and 320° C., particularly preferably between 240 and 300° C. and especially preferably between 245 and 275° C. The pressure is not subject to any restrictions. A reduced pressure is advantageous for safety reasons.

Both acidic and alkaline catalysts are suitable as dehydration catalysts. Acidic catalysts are particularly preferred due to the low tendency to oligomer formation. The dehydration catalyst can be used either as a homogeneous catalyst or as a heterogeneous catalyst.

If the dehydration catalyst is present in the gas phase dehydration as a heterogeneous catalyst, it is preferable that the dehydration catalyst is in contact with a support. Suitable supports are all solids that are apparently suitable to those skilled in the art. In this context, it is preferable that these solids have suitable pore volumes which are suitable for good binding and uptake of the dehydration catalyst. In addition, total pore volumes in accordance with DIN 66133 are preferably in a range from 0.01 to 3 mL/g and particularly preferably in a range from 0.1 to 1.5 mL/g. It is also preferred that the solids suitable as supports have a surface area in the range from 0.001 to 1000 m²/g, preferably in the range from 0.005 to 450 m²/g and more preferably in the range from 0.01 to 300 m²/g according to the BET test in accordance with DIN 66131.

It is possible to use a bulk material as support for the dehydration catalyst having an average particle diameter in the range from 0.1 to 40 mm, preferably in the range from 1 to 10 mm and more preferably in the range from 1.5 to 5 mm. The wall of the dehydration reactor may also serve as support. Furthermore, the support itself may be acidic or basic or an acidic or basic dehydration catalyst can be applied to an inert support. Application techniques particularly include dipping or impregnating or incorporation into a support matrix.

Suitable supports, which may also serve as dehydration catalyst, are in particular natural or synthetic siliceous substances such as in particular mordenite, montmorillonite, acidic zeolites, acidic aluminum oxides, γ-Al₂O₃; monobasic, dibasic or polybasic inorganic acids, particularly phosphoric acid, or support materials covered by acidic salts of inorganic acids, such as oxidic or siliceous substances, for example Al₂O₃, TiO₂; oxides and mixed oxides, such as γ-Al₂O₃ and ZnO—Al₂O₃ mixed oxides of heteropolyacids.

The support preferably comprises, at least in part, an oxidic compound. Oxidic compounds of this kind should comprise at least one of the elements Si, Ti, Zr, Al, P or a combination of at least two thereof. Supports of this kind can also themselves act as dehydration catalyst by virtue of their acidic or basic properties. A preferred compound class, acting both as support and as dehydration catalyst, are silicon-aluminum-phosphorus oxides. Preferred basic substances that function both as dehydration catalyst and as support include alkali metals, alkaline earth metals, lanthanum, lanthanoids or a corm bination of at least two thereof in their oxidic form such as, for example, substances containing Li₂O, Na₂O, K₂O, Cs₂O, MgO, CaO, SrO or BaO or La₂O₃. Such acidic or basic dehydration catalysts are commercially available. A further class are ion exchangers. These may be either in basic or acidic form.

Suitable as homogeneous dehydration catalysts are particularly inorganic acids, preferably acids containing phosphorus and more preferably phosphoric acid. These inorganic acids can be immobilized on the support by dipping or impregnation.

Following the dehydration, a phase containing acrylic acid is obtained which can optionally be purified by further purification steps, in particular by distillation methods, extraction methods or crystallization methods or by a combination of these methods. Further information regarding dehydration in the gas phase can be found in WO 2008/023039 A1.

### EXAMPLES

Reaction of Ethylene Oxide with Carbon Monoxide to Give Poly-3-Hydroxypropionate

The reaction of ethylene oxide with carbon monoxide was carried out in a stirrable autoclave having a blade stirrer (the motion of the blade stirrer was effected via a magnetic coupling), the reaction chamber of which could be optionally heated or cooled externally. All contact surfaces of the reaction chamber were manufactured from Hastelloy HC4. The reaction chamber of the autoclave had a circular cylindrical geometry. The height of the circular cylinder was 335 mm. The internal diameter of the circular cylinder was 107 mm. The envelope of the reaction chamber had a wall thickness of 19 mm (Hastelloy HC4). The top of the autoclave was equipped with a gas inlet-gas outlet valve which opened into the reaction chamber. The temperature in the reaction chamber was determined with the aid of a thermocouple. The reaction temperature was regulated electronically. The internal pressure in the reaction chamber was monitored continuously with an appropriate sensor.

The reaction chamber of the autoclave was initially inertized with argon (Ar content: ≥99.999% by volume Ar, ≤2 ppm by volume O₂, ≤3 ppm by volume H₂O and ≤0.5 ppm by volume of total amount of hydrocarbons).

The autoclave temperature-controlled at 10° C. under argon was then filled with dicobalt octacarbonyl (Co₂(CO)₈); Sigma-Aldrich; specification: 1-10% hexane, ≥90% Co, order number: 60811), 3-hydroxypyridine (Sigma-Aldrich; specification: content 99%, order number: H57009) and diglyme (Sigma-Aldrich; specification: content 99%, order number: M1402) or diethyl phthalate (DEP, supplier: Alfa Aesar; specification: content 99%, order number: A17529) in the ratios specified in Table 1 and the autoclave was then sealed. The temperature of the two solids was 25° C. and the temperature of the diglyme or DEP was 10° C. Then, while maintaining the internal temperature at 10° C., carbon monoxide was pressurized into the autoclave via the valve, until the pressure in the reaction chamber was 1.5° 10⁶ Pa (carbon monoxide from BASF SE, specification: 99.2% CO), Subsequently, the temperature in the reaction chamber was increased to 35° C. in order to verify that the autoclave was gas-tight (over a period of 90 min). The atmosphere in the reaction chamber was then depressurized to an internal pressure of 10⁶ Pa by opening the valve. The temperature in the interior afterwards was 30° C.

Subsequently, ethylene oxide (1.5 g/min) in the amount specified in Table 1 was pumped into the reaction chamber through the valve (supplier: BASF SE; specification: 99.9% purity, 100 ppm water, 50 ppm acetaldehyde and 20 ppm acetic acid). The temperature in the reaction chamber reduced here to 25° C. Subsequently, further carbon monoxide was pressurized into the autoclave until the pressure in the reaction chamber reached 6.10⁶ Pa (while maintaining the internal temperature of 25′C).

The temperature in the reaction chamber of the autoclave was then increased in an essentially linear manner over a period of 45 min to 75° C. while stirring (700 rpm). This temperature was maintained for 8 h while stirring. The pressure in the reaction chamber fell over this period to 5.10⁶ Pa. The heating of the autoclave was then switched off. Within 5 h and 50 min the temperature in the stirred reaction chamber fell essentially exponentially to 25° C. (after 50 min the internal temperature was 60° C., after 150 min it fell to 40° C. and after 235 min to 30° C.). The associated pressure in the reaction chamber was 4.3.10⁶ Pa. The autoclave was then vented to standard pressure and the reaction chamber was purged successively three times with argon (10⁶ Pa).

A dark red-brown solution was in the reaction chamber. This was removed from the autoclave.

300 g of the solution obtained (about 12% by weight poly-3-HP solution with 78% by weight diglyme or DEP as solvent, see Table 1) were initially charged in a 1 L glass reactor equipped with a 2-stage pitched blade stirrer. The reactor was heated by a double-jacketed heat exchanger to 45° C. and maintained at this temperature. To the poly-3HP solution was added the respective antisolvent at a temperature of 20° C. (12.5% acetic acid) in the ratio specified in Table 1. The addition was carried out with stirring at about 605 rpm (corresponding to a power input of 1 W per kg after complete addition of the antisolvent). At the end of the addition, the reactor content was stirred at 605 rpm for a further 10 minutes.

The reactor content was then purged with air (12 L/h) for 0.5 h, wherein the guide tube was positioned below the surface in the vicinity of the extent of the stirrer; at the same time the headspace of the reactor was purged with nitrogen (20° C., 700 L/min). The temperature was 45° C. The resulting suspension was stirred for a further 10 minutes at the same stirrer speed. Subsequently the reaction mixture was allowed to cool without stirring and allowed to stand up to ca. 18 h.

For the solid-liquid separation, a laboratory pressure Nutsche filter was used. The filter medium 42-1100-SK012, Sefar (PTFE, air permeability: 7 L/(dm²·min)) having a filter surface area of 20 cm² was placed on a perforated supporting plate (lower part of the Nutsche filter). The lower part and the cylindrical housing of the Nutsche filter (V is 0.32 L/0.52 L) were attached. A filter container was placed on a balance below the filter outlet. The Nutsche filter was filled with the suspension (mₛᵤₛₚ is ca. 250 to 450 g). The experiment was conducted at room temperature (ca. 20° C.), the precise temperature being noted during the filtration. The Nutsche filter was not temperature-controlled. The Nutsche filter was sealed by connecting the head piece (which comprised the gas inlet opening, a pressure manometer and a pressure release valve) to the main part of the Nutsche filter. The filtration was carried out by applying nitrogen gas pressure (Δp is 2 bar). The filtration was complete as soon as nitrogen passed through the filter medium.

Deionized water was added as wash liquid to the filter cake. The Nutsche filter was closed again and the wash liquid was pressed through the filter cake by applying nitrogen gas pressure (Δp is 2 bar). The pH of the wash filtrate was noted during the wash process. After the wash, nitrogen gas (100 L/h) was passed through the Nutsche filter for 120 seconds in order to dehumidify the filter cake mechanically.

The filter cake was dried in a vacuum drying oven at 10 mbar and 60° C. for three days. The filter cake was weighed before and after drying in order to calculate the dry mass content and the yield. The yield was defined as the mass ratio between the dried filter cake collected and the expected amount of poly-3HP starting from 100% theoretical conversion of the carbonylation reaction.

The cobalt content in the dried product was determined by atomic emission spectroscopy (Varian 720-ES ICP-OES spectrometer, cobalt line 237.86 nm). The molecular weights of the poly-3HP products were analyzed by gel permeation chromatography (GPC, also known as size exclusion chromatography, SEC), in which polymethyl methacrylate was used as standard in order to characterize the average chain length and the dispersity of the molecular weight of the poly-3HP molecules. For the examples listed, a weight average M_{w} was found in the range from 4880 to 7690 g/mol with a dispersity of the molecular weight in the range from 1.7 to 2.3.

For a second decobalting, 89.4 g of dried product were suspended in 190.0 g of acetic acid (12.5%) and heated with nitrogen sparging to 82° C. (internal temperature). The solid of the suspension melted from 61° C. and was present as a dark brown melt phase. After the internal temperature of 82° C. was reached, it was sparged with air (10 L/h) for 30 minutes. The mixture was then cooled under nitrogen. The phases were separated (melt phase: 116.6 g, residue: 152.6 g) and the melt phase was dried at 60° C. for 3 days (77.5 g of poly-3HP, 87% yield). The cobalt content was 0.014 g per 100 g total amount and the weight average M_{w} was 4880 g/mol with a dispersity of the molecular weight of 2.1.

The results are listed in Table 1.

TABLE 1

| Poly- | EO* | Co₂(CO)₈ | S | Co-cat.** | Output: | Yield | Co content |
|---|---|---|---|---|---|---|---|
| 3HP # | [g] | [g] | [g] | [g] | antisolvent | [%] | [wt %] |
| 1 | 98.2 | 16.1 | 946.5^{dig} | 8.7 | 1:5 | 51 | 0.11 |
| 2 | 100.0 | 16.0 | 955.5^{dig} | 8.7 | 1:2 | 78 | 0.58 |
| 3 | 100.0 | 16.1 | 946.8^{dig} | 8.8 | 1:2 | 67 | 0.88/ |
| | | | | | | | 0.040*** |
| 4 | 40.0 | 1.6 | 17.4^{dig} | 0.9 | 1:1 | 69*** | 0.022*** |
| 5 | Mixture of poly-3HP batches, prepared as #2 | | | | | 84*** | 0.073*** |
| 6 | 40.0 | 1.6 | 17.7^{dig} | 0.9 | 1:1.5 | 42 | 0.014 |
| 7 | Mixture of poly-3HP batches, prepared as #2 | | | | | 50-75 | 0.033 |
| 8 | 99.8 | 8.0 | 960.0^{DEP} | 4.4 | 1:2.5 | 46 | 0.001 |
| 9 | 100.0 | 8.0 | 960.0^{DEP} | 4.4 | 1:2**** | 42 | 0.003 |
| 10 | 100.0 | 8.0 | 960.0^{DEP} | 4.4 | 1:1**** | 75 | 0.062 |

*EO = ethylene oxide;
**Co-cat. = 3-hydroxypyridine;
***after second decobalting;
****75° C. instead of 45° C.;
S (solvent): dig = diglyme, DEP = diethyl phthalate;
antisolvent: 12.5% acetic acid

Transesterification

### Examples 1 & 2

The influence of the cobalt content of the poly-3HP on the transesterification was investigated. For this purpose, a poly-3HP batch with 0.3% by weight cobalt was compared with a poly-3HP batch with 0.040% by weight cobalt. To the respective poly-3HP batches in the amounts specified in Table 2 was added a tenfold amount of butanol and the mixture was heated at 170° C. in a 300 mL autoclave for 8 h. The pressure in the autoclave was in the range from 6 to 7 bar.

The respective yield of butyl 3-hydroxypropionate was determined by GC (internal standard: decane). The results are listed in Table 2.

It is evident from Examples 1 and 2 that with increasing cobalt content in the poly-3HP a distinctly higher yield can be achieved in the esterification.

### Examples 3 to 7

In a glass flask connected to a water separator (Examples 3 to 5) or an autoclave (Examples 6, pressure 5.5 bar and Example 7, pressure: 8 bar), poly-3HP, an alcohol and optionally a solvent, in the ratios specified in Table 2, were heated to 162 to 170° C. for 7 to 14 h. The respective yield of 3-hydroxypropionic acid alkyl ester was determined by GC (internal standard: decane, for the calibration butyl 3-hydroxypropionate was used). The results are listed in Table 2.

It is evident from Examples 3 to 7 that the reaction in the target ester is linked to an increased yield. An additional high boiler such as tetraglyme or dodecane has no advantage compared to the reaction in pure alcohol.

### Examples 8 to 12

In a glass flask connected to a water separator, poly-3HP, n-hexanol and Al(III) mesylate were heated to 154 to 166° C. for 3 to 9.3 hours, in the ratios specified in Table 2. The respective yield of hexyl 3-hydroxypropionate was determined by GC (internal standard: decane, for the calibration butyl 3-hydroxypropionate was used). The results are listed in Table 2. No by-products were formed.

It is evident from Examples 8 to 12 that Al(III) mesylate as transesterification catalyst produces a distinctly higher conversion. A higher cobalt content in turn produces higher yields.

### Examples 13 to 21

Al(III) mesylate, Mg(II) mesylate, Ti(IV) mesylate, Ti(IV) n-butoxide, Al(III) isopropoxide and Al(III) acetate (basic) were investigated for their catalytic activity in the transesterification of poly-3HP. The mesylates themselves were prepared; the remaining compounds were obtained from Sigma Aldrich.

In a glass flask connected to a water separator, 3 g of poly-3HP, 60 g of n-hexanol and optionally 30 mg of catalyst were heated to 154 to 164° C. for 0.5 to 8 hours, in the ratios specified in Table 2. The respective yield of hexyl 3-hydroxypropionate was determined by GC (internal standard: decane, for the calibration hexyl 3-hydroxypropionate was used).

Complete conversion to hexyl 3-hydroxypropionate was achieved after 4 hours with Al(III) mesylate (Example 14), In comparison thereto, in the uncatalyzed transesterification, only 8.3% hexyl 3-hydroxypropionate was formed within 4 hours.

Methanesulfonic acid, which can be formed in the hydrolysis of Al(III) or Ti(IV) mesylate, exhibited no significant catalytic activity (Example 16). In the case of Ti(IV) butoxide (Example 19), the product concentration dropped after four hours; GC analysis indicated the formation of acrylate due to dehydration of the product.

It is evident from Examples 13 to 21 that various catalysts can be used in order to achieve a high, sometimes quantitative conversion.

### Examples 22 to 27

The transesterification of poly-3HP with n-butanol, optionally in the presence of a catalyst, was investigated. Owing to the relatively low boiling point of n-butanol (118° C.), most of the experiments were conducted in the autoclave in order to achieve the activation temperature of the esterification (here about 125° C.).

In a glass flask connected to a water separator, 20 g of poly-3HP, 100 g of n-butanol and 200 mg of Al(III) mesylate were heated to 116 to 123° C. for 22 to 30 hours (Example 22). Poly-3HP, n-butanol and optionally a catalyst, in the ratios specified in Table 2, were heated to 170° C. in a 300 mL autoclave for 8 hours (Examples 23 to 27). The pressure in the autoclave was 4 to 5 bar. The respective yield of butyl 3-hydroxypropionate was determined by GC (internal standard: decane).

It is evident from Examples 22 to 27 that in esterifications in which the boiling point of the alcohol is below the activation temperature for the esterification, this fact can be taken into account by using an autoclave.

### Examples 28 to 49

The transesterification of poly-3HP with various alcohols, optionally in the presence of a catalyst, were investigated in glass flasks or in the autoclave.

Poly-3HP, an alcohol and optionally a catalyst, in the ratios specified in Table 2, were heated to 119 to 180° C. in a glass flask attached to a water separator or a 300 mL autoclave (pressure: 2 bar for 1-dodecanol to 22 bar for methanol) for 4 to 22 hours. The respective yield of 3-hydroxypropionic acid alkyl ester was determined by GC (internal standard: decane, for the calibration butyl 3-hydroxypropionate was used). In the absence of a catalyst, methyl acrylate was detected in the transesterification in methanol.

It is evident from Examples 28 to 49 that numerous alcohols can be used in the esterification. The use of the catalyst always leads to improved yields of the respective alkyl ester.

TABLE 2

| | | | Cobalt | | | | | |
|---|---|---|---|---|---|---|---|---|
| | | Poly-3HP | content | | | | | Yield |
| Example # | Poly-3HP # | amount | [wt %] | Alcohol | Catalyst | Temp. | Time | Ester |
| 1* | 1 | 10 g | 0.3 | 100 g n- | — | 170° C. | 8 h | 71.7% |
| | | | | butanol | | | | |
| 2* | 3 | 11 g | 0.040 | 110 g n- | — | 170° C. | 8 h | 38.6% |
| | | | | butanol | | | | |
| 3 | 4 | 2.3 g | 0.022 | 6 g n-hexanol | — | 170° C. | 8 h | 16.0% |
| | | | | in 54 g tetraglyme | | | | |
| 4 | 4 | 2.3 g | 0.022 | 6 g n-hexanol | — | 170° C. | 8 h | 10.4% |
| | | | | in 54 g dodecane | | | | |
| 5 | 4 | 10 g | 0.022 | 100 g n- | — | 162° C. | 7 h | 33.5% |
| | | | | hexanol | | | 14 h | 44.4% |
| 6* | 3 | 10 g | 0.040 | 100 g n- | — | 170° C. | 14 h | 13.3% |
| | | | | butanol | | | | |
| 7* | 3 | 12 g | 0.073 | 100 g n- | — | 170° C. | 14 h | 13.5% |
| | | | | hexanol | | | | |
| 8 | 4 | 3 g | 0.022 | 60 g n- | 30 mg Al(III) mesylate | 163° C. | 3 h | 67.9% |
| | | | | hexanol | | | | |
| 9 | 4 | 10 g | 0.022 | 100 g n- | 100 mg Al(III) mesylate | 165° C. | 3 h | 71.1% |
| | | | | hexanol | | | 5 h | 71.9% |
| 10 | 6 | 10 g | 0.014 | 100 g n- | 100 mg Al(III) mesylate | 166° C. | 3 h | 21.3% |
| | | | | hexanol | | | 5 h | 42.0% |
| | | | | | | | 9.3 h | 61.2% |
| 11 | 4 | 10 g | 0.022 | 100 g n- | 100 mg Al(III) mesylate | 166° C. | 5 h | 70.2% |
| | | | | hexanol | | | | |
| 12 | 7 | 3 g | 0.033 | 60 g n- | 30 mg Al(III) mesylate | 154-162° C. | 6.5 h | 75.1% |
| | | | | hexanol | | | | |
| 13 | 4 | 3 g | 0.022 | 60 g n- | 30 mg Ti(IV) mesylate | 154-162° C. | 0.5 h | 70.5% |
| | | | | hexanol | | | 1 h | 94.3% |
| | | | | | | | 4 h | 95.1% |
| 14 | 4 | 3 g | 0.022 | 60 g n- | 30 mg Al(III) mesylate | 154-162° C. | 1 h | 18.3% |
| | | | | hexanol | | | 4 h | 100.7% |
| 15 | 4 | 3 g | 0.022 | 60 g n- | — | 154-158° C. | 4 h | 8.3% |
| | | | | hexanol | | | 8 h | 14.2% |
| 16 | 4 | 3 g | 0.022 | 60 g n- | 30 mg methylsulfonic acid | 154-160° C. | 4 h | 8.6% |
| | | | | hexanol | | | 7 h | 11.6% |
| 17 | 7 | 3 g | 0.033 | 60 g n- | 30 mg Al(III) acetate (basic) | 154-163° C. | 4 h | 41.8% |
| | | | | hexanol | | | 7 h | 78.8% |
| 18 | 4 | 3 g | 0.022 | 60 g n- | 30 mg Mg(II) mesylate | 154-160° C. | 4.5 h | 13.8% |
| | | | | hexanol | | | 7 h | 19.6% |
| 19 | 7 | 3 g | 0.033 | 60 g n- | 30 mg Ti(IV) butoxide | 154-162° C. | 0.5 h | 99.3% |
| | | | | hexanol | | | 1 h | 99.6% |
| | | | | | | | 4 h | 96.4% |
| 20 | 7 | 3 g | 0.033 | 60 g n- | 30 mg Co(II) acetate | 154-161° C. | 4 h | 87.5% |
| | | | | hexanol | | | 5 h | 100.1% |
| | | | | | | | 7 h | 88.5% |
| 21 | 7 | 3 g | 0.033 | 60 g n- | 30 mg Al(III) isopropoxide | 154-164° C. | 3.5 h | 40.0% |
| | | | | hexanol | | | 6 h | 78.2% |
| 22 | 5 | 20 g | 0.073 | 100 g n- | 200 mg Al(III) mesylate | 116-123° C. | 22 h | 13.0% |
| | | | | butanol | | | 30 h | 41.0% |
| 23* | 5 | 20 g | 0.073 | 100 g n- | 200 mg Al(III) mesylate | 170° C. | 8 h | 85.2% |
| | | | | butanol | | | | |
| 24* | 5 | 20 g | 0.073 | 100 g n- | 200 mg Al(III) mesylate | 170° C. | 8 h | 85.9% |
| | | | | butanol | | | | |
| 25* | 7 | 5 g | 0.033 | 50 g n- | 50 mg Al(III) mesylate | 170° C. | 4 h | 88.1% |
| | | | | butanol | | | | |
| 26* | 7 | 5 g | 0.033 | 50 g n- | — | 170° C. | 4 h | 14.3% |
| | | | | butanol | | | | |
| 27* | 7 | 5 g | 0.033 | 50 g n- | 50 mg Co(II) acetate | 170° C. | 4 h | 75.7% |
| | | | | butanol | | | 8 h | 77.8% |
| 28* | 4 | 12 g | 0.022 | 100 g methanol | — | 170° C. | 8 h | 33.9% |
| 29* | 4 | 12 g | 0.022 | 100 g methanol | — | 170° C. | 36 h | 35.2 |
| 30* | 5 | 12 g | 0.073 | 100 g methanol | — | 170° C. | 14 h | 45.3% |
| 31* | 8 | 3 g | 0.001 | 60 g methanol | 30 mg Al(II) mesylate | 170° C. | 4 h | 44.9% |
| 32* | 3 | 2 g | 0.040 | 20 g ethanol | — | 170° C. | 8 h | 31.0% |
| 33* | 8 | 3 g | 0.001 | 60 g ethanol | 30 mg Al(III) mesylate | 170° C. | 4 h | 38.0% |
| 35* | 3 | 2 g | 0.040 | 20 g n- | — | 170° C. | 8 h | 21.6% |
| | | | | butanol | | | | |
| 36 | 3 | 5 g | 0.040 | 100 g n- | — | 119° C. | 10 h | 2.2% |
| | | | | butanol | | | | |
| 37* | 3 | 5 g | 0.040 | 100 g n- | — | 170° C. | 8 h | 37.3% |
| | | | | butanol | | | | |
| 38* | 3 | 11 g | 0.040 | 110 g n- | — | 170° C. | 8 h | 38.6% |
| | | | | butanol | | | | |
| 39* | 5 | 20 g | 0.073 | 100 g n- | 200 mg Al(III) mesylate | 170° C. | 8 h | 85.9% |
| | | | | butanol | | | | |
| 40* | 3 | 2 g | 0.040 | 20 g n- | — | 170° C. | 8 h | 31.4% |
| | | | | hexanol | | | | |
| 41 | 3 | 10 g | 0.040 | 100 g n- | — | 162° C. | 14 h | 22.9% |
| | | | | hexanol | | | | |
| 42 | 3 | 12 g | 0.040 | 120 g n- | — | 162° C. | 14 h | 31.2% |
| | | | | hexanol | | | | |
| 43* | 3 | 2 g | 0.040 | 20 g cyclohexanol | — | 170° C. | 8 h | 9.1% |
| 44 | 3 | 3 g | 0.040 | 60 g cyclohexanol | — | 180° C. | 21 h | 17.8% |
| 45 | 7 | 3 g | 0.033 | 65 g cyclohexanol | 30 mg Al(III) mesylate | 160-167° C. | 21 h | 65.5% |
| 46* | 3 | 2 g | 0.040 | 20 g n-octanol | — | 170° C. | 8 h | 30.8% |
| 47 | 3 | 3 g | 0.040 | 60 g n-octanol | — | 170° C. | 14 h | 51.0% |
| 48 | 3 | 3 g | 0.040 | 60 g 1-dodecanol | — | 170° C. | 14 h | 33.2% |
| 49 | 7 | 3 g | 0.033 | 60 g 1-dodecanol | 30 mg Al(III) mesylate | 170° C. | 22 h | 56.2% |

*carried out in the autoclave

### Dehydration

### Example 50

30 g (205 mmol) of butyl 3-hydroxypropionate in 30 g of n-butanol (50 wt % solution) were converted in the presence of 30 mL (161 g; 1.5 mm strands) of aluminum oxide (BASF D10-10) to give butyl acrylate. The reaction was conducted at a pressure of 1 bar (standard pressure) and a temperature of 250° C. in a conventional fixed bed gas phase reactor. The feed solution was passed through the reactor at 10 mL/h, while at the same time nitrogen was passed through the reactor at 10 NL/h. The product was condensed into a flask, which comprised 10 mg of 4-methoxyphenol in 79.5 g of n-butanol as stabilizer.

The yield of butyl acrylate was determined by GC (internal standard: diethylene glycol dibutyl ether) and was 75%.

### Comparative Example 1

The suitability of sulfuric acid as transesterification catalyst was investigated (as in WO 2013/185009 A1). To 1.2 g of poly-3HP (98%, 0.022 wt % cobalt, weight average M_{w}=7690 g/mol and dispersity 2.3) in 81 mL of butanol in a glass flask were added 4 g of sulfuric acid and the mixture was heated to 117° C. for 21 h. By means of GC-MS, dibutyl ether, dibutyl sulfate and butyl 3-butoxypropanoate could be detected as products, but no butyl acrylate.

1.-12. (canceled)

13. A method for preparing a 3-hydroxypropionic acid ester, comprising
a) reacting ethylene oxide with carbon monoxide in the presence of a cobalt catalyst, wherein poly-3-hydroxypropionate is obtained;
b) transesterifying the poly-3-hydroxypropionate with an alcohol in the presence of a transesterification catalyst, wherein the 3-hydroxypropionic acid ester is obtained;
wherein the transesterification catalyst is a compound of the formula
MLₓ (I)
where
M is a metal of main groups 2, 3 or 4 or transition groups 3 to 8 of the Periodic Table of the Elements,
L is a ligand, which is bonded directly to M via a C, an O, a P, an S and/or an N atom, and
x is an integer from 2 to 6.

14. The method according to claim 13, wherein M is titanium, aluminum or cobalt.

15. The method according to claim 13, wherein L is an alkyl, an alkoxy, an alkylcarboxyl, an alkylsulfoxy or an aryl radical.

16. The method according to claim 13, wherein the transesterification catalyst is Ti(IV) mesylate, Ti(IV) butoxide, Al(III) mesylate, Al(III) isopropoxide, Al(III) acetate, Al(III) hydroxyacetate or Co(II) acetate.

17. The method according to claim 13, wherein reaction a) is carried out in an aprotic solvent, and poly-3-hydroxypropionate is precipitated from the resulting solution of poly-3-hydroxypropionate in the aprotic solvent by adding an aqueous antisolvent.

18. The method according to claim 13, wherein the alcohol is selected from C₁₋₁₈-alcohols.

19. The method according to claim 18, wherein the alcohol is selected from the group consisting of methanol, ethanol, cyclohexanol, n-butanol and 2-ethylhexanol.

20. The method according to claim 13, wherein the transesterification b) is conducted at a temperature of 130 to 200° C.

21. The method according to claim 13, wherein a mixture is obtained in the preparation/mixture a) from which poly-3-hydroxypropionate is precipitated or is obtained as a melt of the liquid-liquid separation.

22. A method for preparing an acrylic acid ester, comprising:
(i) preparing a 3-hydroxypropionic acid ester by the method according to claim 13; and
(ii) dehydrating the 3-hydroxypropionic acid ester.

23. The method according to claim 22, wherein the dehydration of the 3-hydroxypropionic acid ester is conducted in the gas phase.

24. The method according to claim 22, wherein the dehydration of the 3-hydroxypropionic acid ester is conducted in the liquid phase.
